(19) United States
(12) Patent Application Publication
(10) Pub. No.: US 2019/0183893 A1
(43) Pub. Date: Jun. 20, 2019

(54) Low dose of sildenafil as an antitumor drug

(71) Applicant: Guangzhou Dawnlight Biotech Company, Guangzhou City, Guangdong Province (CN)

(72) Inventor: Yu Liu, Guangzhou City, Guangdong Province (CN)

(21) Appl. No.: 15/759,575

(22) PCT Filed: Oct. 31, 2016
(86) PCT No.: PCT/CN2016/103957

§ 371 Date: Mar. 13, 2018

(30) Foreign Application Priority Data
Oct. 9, 2016 (CN) 201610884154.0

(51) Int. Cl. A61K 31/519; A61P 35/00; A61K 9/00

(52) U.S. Cl. CPC A61K 31/519; A61K 9/0053; A61K 9/0019; A61P 35/00

(57) ABSTRACT

The present invention is related to the medical technology field. This invention discloses the application of sildenafil at low doses as an antitumor drug, with the chemical name of 1-[4-ethyl-3-[5-(6,7-Two-methyl-7-oxygen generation-3-propyl H pyrazole and [4,3d] pyrimidine)] phenyl sulfonyl-4-biphenyl methyl piperazine, and the use dosage is: the oral dosage of 0.04˜0.5 mg per 1 kg of body weight, or 2˜25 mg for an adult with body weight of 50 kg, or injection dosage of 0.04˜0.2 mg per 1 kg of body weight, or 2˜10 mg for an adult with body weight of 50 kg, or external dosage in a mass percentage of less than 1% of sildenafil. This invention is applicable to the treatment of malignant tumors. It inhibits tumor growth by more than 50% without significant toxic or side effects, and thereby, sildenafil can be used in control of tumor growth and for improvement of other treatments of tumor.

### TECHNICAL FIELD

The invention, which relates to the technical field of medicine, specifically refers to the application of low doses of sildenafil as an antineoplastic agent.

### BACKGROUND TECHNOLOGY

Malignant tumor, also known as cancer, can take place in most organs of the body, with its main clinical features which are rapid development, strong infiltration of the organization, transferability to other parts of the body, ability to produce harmful substances to damage the normal structure of organs and to throw body dysfunction out of gear, and to threaten the lives of patients. According to the latest data of WTO, the global incidence of cancer will increase by 50% till the year of 2020, that is, 15 million new cancer patients will be added each year. Worse still, the number of deaths from cancer is also rising sharply all over the world. A total number of 7.6 million people died of cancer in 2007, this number may increase to 13.2 million in 2030. Moreover, 20% of the new cancer patients and 24% of the cancer deaths are in China. At present, 1 person died of cancer in every 5 deaths in China, and 1 person died of cancer in every 4 deaths through the population of 0-64 years old. In the “Chinese Annual Cancer Registry Review of 2012”, 6 patients can be diagnosed with cancer every minute in China, which means, 8,000 patients can be diagnosed with cancer every day, and more than 3 million new cancer patients every year.

There is not yet a particularly effective antineoplastic drug. Compared with the huge number of cancer patients, there are limited options of antineoplastic drugs but all at expansive prices, and therefore, a large number of cancer patients cannot be effectively treated. Traditional western medicine treatment for cancer is mainly chemotherapy, surgery and radiotherapy, while there are still problems to be solved such as strong side effects, high metastasis and recurrence rates. Therefore, many countries tend to develop targeting drugs and immune cell treatment technology at present for higher efficacies. “Targeting drugs” are therapeutic techniques that direct the killing or inhibition of cancer cells but rarely harm normal cells, which have become an important direction in the study of new technologies for cancer treatment. “Targeting drugs” can be divided into the followings according to their roles on different targets:

(1) Gene therapy and viral therapy: as early as 2004, more than 1,020 programs were made for clinic use and 63.4% of which were used in cancer treatment. The main result was the transport of antiangiogenic factor, tumor suppressor gene, precursor drug activation gene and immune stimulating gene. Among them, P53 gene carried by adenovirus was the most rapid development in clinic, with at least 5 programs entering the III phase of clinical trials in the world. However, there are still many obstacles in tumor gene therapy, the main problem is that the carrier can not specifically target the tumor cells, treatment of the gene in tumor cells at high level expression is not enough to eliminate tumor cells, and virus carrier shell changes. (2) Antibody therapy: in recent years, a breakthrough has been made in the study of antibody drugs for the treatment of tumors. At present, more than 500 kinds of antibodies have been used for diagnosis and treatment in the world, and FDA has approved 18 antibody listings, of which 8 are targeting antibodies for tumor therapy, for example, in the treatment of lymphoma antibody Rituxan has treated more than 300,000 patients; the total response rate for first-line treatment was 60%˜75%, and its efficacy was the same as chemotherapy. The effective rate if combined with chemotherapy was as high as 80%, the total remission rate was up to 40%˜63%, and the antibody Avastin against vascular endothelial growth factor could prolong the survival period of patients with advanced colon cancer by an average of 5 months, which is currently used in 95% colon cancer patients in the United States. However, there are still 3 problems with antibody therapy for solid tumors: the antibody is difficult to penetrate the solid tumor cells, so the treatment of massive solid tumors is still not satisfactory; production costs and prices are very expensive; due to only a specific receptor for tumor cells, the treatment needs to be labeled with isotopes or toxins, but the side effects also increased. (3) Signal conduction pathway therapy: the occurrence and development of tumor are closely related to the cell proliferation, apoptosis and other signal transduction pathways. One of the most important molecules in signal transduction is protein tyrosine kinase, which is one of the research hotspots for antineoplastic drugs and targeting drug development. Small molecule STI-571 (Gleevec or Glivec), which has been approved by FDA, can kill tumor cells specifically. Over the past 15 years, a number of small peptides have been found that they can be worn into living cells, and these peptides carry exogenous substances into the cells and are highly efficient. However, at present, these methods are still in the preliminary experiments, because there are many problems to be solved, such as the membrane-piercing peptide that can not be pierced into all cells, its penetration mechanism is not clear, and it induces immune responses which will interfere with its efficacy in vivo. (4) RNA interference technique: RNA interference (Rnainterference, RNAi) is a kind of mRNA degradation induced by short double strain RNA. This phenomenon occurs at post transcription levels, also known as post transcription gene silencing. RNAi only degrades the mRNA of a single endogenous gene corresponding to the sequence, which has high specificity and efficiency. The transplanted tumor model with small interfering RNA gene drug therapy can improve the sensitivity of chemotherapeutic drugs at the same time. However, RNAi treatment technology has not been widely used, since the main problem is that RNAi is not easy to be introduced to tumor tissues, and in vivo the half-life is relatively short. (5) Small molecule targeting drugs: the development of new drugs targeting protein tyrosine kinase is progressing rapidly. This kinase can catalyze the transfer of gamma phosphate in ATP to tyrosine residues of a variety of proteins, which plays an important role in the process of cell growth, proliferation and differentiation. Herceptintm (Genentech and Roche) is a human-derived monoclonal antibody with tyrosine kinase receptor her2/neu as the target, and has additive or synergistic effects with a variety of chemotherapeutic drugs. GLEEVECTM (Swiss Novatis) is a specific inhibitor of tyrosine kinase bcrabl, which has a very good effect on the treatment of chronic myeloid leukemia, and has been listed in advance by the FDA for the treatment of patients with chronic myeloid leukemia with a positive chromosome in Philadelphia. Iressa (AstraZeneca) is an oral small molecule inhibitor for EGFR tyrosine kinase. FDA approved its use in Advanced non-small cell lung cancer (NSCLC) for chemotherapy failure with platinum or yew regimens in May 2003, and it is the 1st small molecule tyrosine kinase inhibitor targeting at specific targets for solid tumor therapy. However, small molecule targeting drugs usually have high toxicity, and there are obvious individual differences, so that most of the small molecule targeting drugs are in the experimental treatment stage. (6) Targeting treatment of viral vectors: recent studies have found that several cells can carry viral vectors for systemic drug therapy. These cells include macrophages, T cells, NK cells, allogeneic tumor cells, and the hottest stem cells currently studied. These tumor chemotaxis cells can induce signals from tumor microenvironment, have the function of tracking tumors and transfer genes, but still need to be studied more deeply. Compared with traditional methods, the use of stem cells to treat disease has the advantages of low toxicity, effective use of one drug, and no need to fully understand the exact mechanism of the disease pathogenesis. However, since mesenchymal stem cells are mainly derived from bone marrow, it is difficult to expand these cells in large numbers after chemotherapy. Immune cell therapy is a biological therapy for the tumor patients to obtain the immunized cells with antineoplastic activity, to kill the tumor directly or to stimulate the body antineoplastic immune response. Its operations include the passage, amplification, modification, screening and treatment of cells in vitro, which can change the biological behavior of the cells, and can be used in the treatment of tumor and tumor prevention in vitro. Somatic cell immunotherapy has become one of the most important methods for adjuvant therapy after radiotherapy and chemotherapy in patients with tumor, which has good effects on the reconstruction of immune system, elimination of residual lesions and bone marrow purification. However, immunotherapy is currently in the primary stage, mainly for adjuvant treatment, and patients have different responses, for instance some patients are not sensitive to this therapy.

Although the present antineoplastic drugs and treatment techniques have some curative effects, there are still some problems, such as high cost, narrow antineoplastic spectrum and large side effects. Therefore, scientists and technicians have continuously tried to search for other antineoplastic drugs, of which the drug for treatment of male erectile dysfunction—sildenafil—is invented for the use in combination with other drugs in antineoplastic treatment with significant effects.

For example, sildenafil (Intraperitoneal injection), combined with Adriamycin (Doxorubicin), can significantly inhibit the growth of transplanted tumor of prostate cancer in nude mouse (PNAS magazine, Page 18202, Volume 107 in 2010), sildenafil (mouth-Fed), combined with Stivarga (regorafenib), can significantly inhibit the growth of transplanted tumor derived from liver cancer in nude mouse (J. Cell. Physiol Magazine, Page 2281, Volume 230 in 2015). These studies have shown that sildenafil can primarily increase the efficacy of other antineoplastic drugs in the treatment of tumors. However, if insildenafil is used alone at normal doses, its antineoplastic effect is not significant (normal dose range: 25-100 mg), and no significant difference is found in comparison with the control group (see above) because of the side effects of the drug. Because the studies did not find out the safe and effective dose-window of sildenafil as an antineopalstic agent, it is at present mainly used in combination with other antineoplastic drugs.

Therefore, it is urgent to search for the safe and effective dosage window for sildenafil as an antineoplastic agent.

### CONTENTS OF THE INVENTION

In view of the current domestic and foreign antineoplastic drugs, mostly they have low efficacy and high toxicity or side effects, etc. In particular, the use normal doses of sildenafil as antineoplastic drug showed no significant efficacy. This invention provides a new technology for sildenafil application: low dosage of sildenafil as an antineoplastic agent, since its use in tumor treatment exhibits good effects but no significant toxicity or side effects. This technique in tumor treatment has remarkable advancement compared to the normal doses of sildenafil.

In order to achieve the above purpose, this invention adopts the following technical schemes to realize: the application of sildenafil of low dosage as antineoplastic drug, the chemical name is 1-[[3-(6,7-Dihydro-1-methyl-7-oxo-3-propyl-1H-pyrazolo[4,3-d]pyrimidin-5-yl)-4-ethoxyphenyl]sulfonyl]-4-methylpiperazine citrate, the use dose of sildenafil is 0.04˜0.5 mg per 1 kg of body weight. Preferably, the method to use sildenafil as an antineoplastic agent includes internal, injection and topical uses.

Preferably, sildenafil as an antineoplastic drug is used in forms of oral solution, powder, tablet, capsule, injection or ointment.

Preferably, the oral dosage of sildenafil as an antineoplastic agent is 0.04˜0.5 mg per 1 kg of body weight. Preferably, the injection dosage of sildenafil as an antineoplastic agent is 0.04˜0.2 mg per 1 kg of body weight. Preferably, the external dosage of sildenafil as an antineoplastic agent is: the mass percentage content of sildenafil is less than 1%. It should be noted that the external dosage is based on the total amount of sildenafil and bases.

Preferably, the topical formulation of sildenafil ointment is as the follows: sildenafil of 0.01˜1% and the bases of 99˜99.99%, which are percentages of the mass, and the bases are lipid-soluble medium such as Vaseline.

Sildenafil as an antineoplastic agent, is administered at a daily oral dose of 0.04˜0.5 mg per 1 kg of body weight, or injected at a single dose of 0.04˜0.2 mg per 1 kg of body weight, or used at topical doses of less than 1% by mass, for the treatment of malignant tumors. Sildenafil via oral use inhibits tumor growth by more than 50% and does not produce significant toxicity or side effects. To achieve the effective control of tumor growth, sildenafil is administered at low doses by the above routes to improve the effective treatment of the tumor.

In mouse transplanted tumor tests of the invention, with sildenafil ethanol solution (dissolved in 25% ethanol) gavage at 10 mg/kg/day, continuous for 36 days, the tumor growth inhibition rate reached 68%, and the mice during the experimental period exhibited no significant or observable toxicity reactions. However, when the dose was 50 mg/kg/day, sildenafil showed no obvious inhibitory effects on growth of the transplanted tumor in mice.

The antineoplastic effect of sildenafil has an optimal dose-window, located between 0.04˜0.5 mg per 1 kg of body weight in humans, or 2-25 mg for an adult with body weight of 50 kg, which is a low dose range for antineoplastic efficacy and safety: the least adverse effects on cancer patients with the best antineoplastic efficacy. Below or above this dose range (0.04˜0.5 mg per 1 kg of body weight in humans, or 2-25 mg for an adult with body weight of 50 kg) will significantly weaken its antineoplastic efficacy. Since this dose range is significantly smaller than the normal dosage of sildenafil for male erectile dysfunction, it is known as “low dose” sildenafil. In comparison with the normal dosage (25-100 mg for an adult), low dose of sildenafil is not only able to significantly improve the antineoplastic efficacy of sildenafil, but also to avoid the side effects of the drug in treatment of cancer patients. Depending on the age, weight and physical conditions of the patient, the optimal dosage of sildenafil as an antineoplastic agent will change accordingly. For example, for the average weight of an adult patient with tumor (weight around 60 kg), the best oral dose of sildenafil is about 10 mg.

The antitneoplastic mechanism of sildenafil at low doses is not yet clear. The experimental results showed that sildenafil at high concentration (200 μM) had a direct inhibitory effect on the growth of cultured tumor cells, for which the mechanism may be the stop growth of tumor cells in the G1 period caused by inhibition of the cell cycle protein (cyclin) and cell cycle-dependent protein kinase (CDK) in the tumor cells. In addition, sildenafil at higher concentration (300 μM) also induced production of reactive oxygen species (ROS) in cultured tumor cells, resulting in tumor cell apoptosis. However, after an adult patient orally takes 100 mg sildenafil citrate tablets, sildenafil in the blood reaches the highest concentration of 440 ng/ml, or 0.66 μM, indicating that sildenafil of this concentration is significantly lower than the above doses required for the antineoplastic mechanism as described. Therefore, the antineoplastic mechanism for low dose sildenafil does not belong to that in tumor cell experiments with high doses of sildenafil.

Further studies also showed that lower concentration (2 μM) of sildenafil stimulated the expression of nuclear factor inhibitory protein IkB nitro-tyrosine and apoptotic gene ligand Fas-L in human hepatocellular carcinoma cell line (HEPG2), thereby promoting tumor cell apoptosis. However, if the low oral doses (2-10 mg) of sildenafil are considered for an adult, the drug concentration in the blood should be lower than 0.1 μM, and the antineoplastic activity of low dose sildenafil may not be the same mechanism through directly inhibiting the growth of tumor cells or promoting tumor cell apoptosis as described above. Therefore, the antineoplastic effects of low dose sildenafil in vivo may not be direct actions of the drug on tumor cells.

In the mouse transplanted tumor experiments, it was demonstrated that the antineoplastic efficacy of low dose sildenafil could be offset by the anti-allergic drug chlorphenamine malate. When the tumor-bearing mice were gavaged with 10 mg/kg/day and 20 mg/kg/day doses of sildenafil citrate respectively for 2 weeks, the tumor inhibition rate was 63% and 50%, respectively. However, when sildenafil citrate of the same doses was combined with chlorphenamine maleate of 2 mg/kg/day dose, the tumor inhibition rate was reduced to 16% and 7%, respectively. It is indicated that chlorphenamine maleate can directly counteract the antineoplastic activity of low dose sildenafil. Therefore, the antineoplastic activity of low dose sildenafil in vivo does not directly affect the tumor cell growth, but it may involve regulation of the immune system by increasing the functions of immune cells to achieve its antineoplastic effects, because the body's immune cells such as lymphocytes, granulocytes and mononuclear cells have H1 receptors which mediate the immune response to pathogens, and H1 receptor antagonist chlorphenamine maleate counteracts the antineoplastic efficacy of low dose sildenafil by suppressing the immune response.

Compared with the prior art, the beneficial effect of the invention is as follows:

The present invention of the new use of low dose Sildenafil citrate, which is used as an antineoplastic agent at a daily oral dose of 0.04˜0.5 mg per 1 kg of body weight, or 2˜25 mg for an adult with body weight of 50 kg, or an injection dose of 0.04˜0.2 mg per 1 kg of body weight, or 2˜10 m for an adult with body weight of 50 kg, or a topical dose of less than 1% by mass weight, is applied to the treatment of malignant tumors, and its inhibition rate of tumor growth in vivo is more than 50% without significant toxic side effects. It can be used to achieve effective control of tumor growth and improve the treatment of tumors.

### DESCRIPTION OF FIGURES

FIG. 1 is a schematic diagram of the chemical structure of sildenafil of the invention;

FIG. 2 is a schematic diagram of the therapeutic dose-window of sildenafil of the invention;

FIG. 3 is a diagram of the inhibitory effects of sildenafil of different doses on transplanted tumor in mice;

FIG. 4 is a schematic diagram of the antagonistic effect of chlorpheniramine malate on the antitumor activity of low-dose sildenafil.

### SPECIFIC MODE OF EXECUTION

The present invention is further described in combination with embodiments, but it should be stated that the embodiments do not limit the scope of protection required by the present invention.

### IMPLEMENTATION EXAMPLE 1

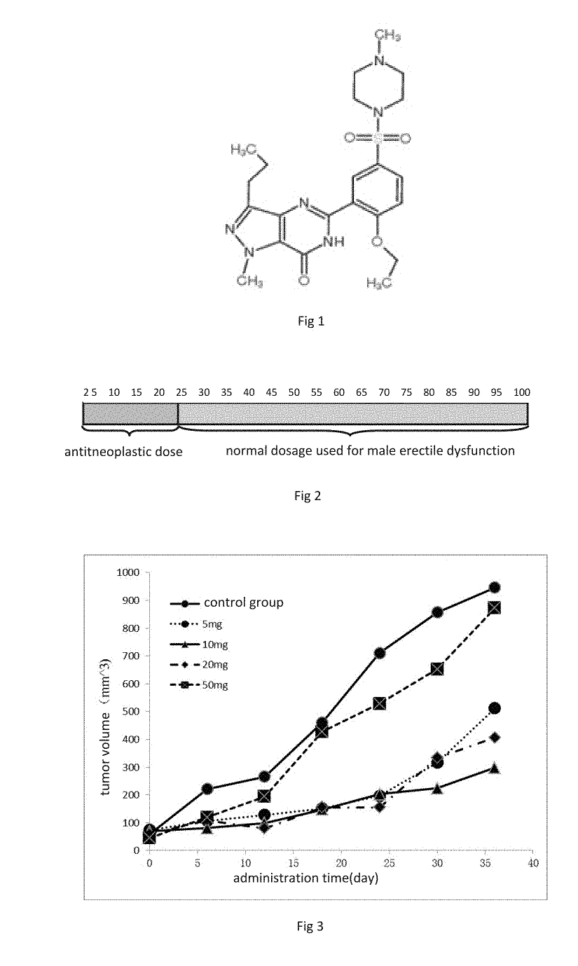

As for the application of sildenafil of a low dose as an antineoplastic agent, the chemical name of mentioned sildenafil is 1-[4-ethyl-3-[5-(6,7-Two-methyl-7-oxygen generation-propyl H pyrazole and [4,3d] pyrimidine)] Benzene sulfonyl-4-biphenyl methyl piperazine citrate. The chemical structure is shown in FIG. 1 and the oral dosage of sildenafil is 0.04˜0.2 mg per 1 kg of body weight, or 2˜25 mg for an adult with body weight of 50 kg. FIG. 2 is a schematic diagram of the antineoplastic dose-window of sildenafil by mg.

For oral administration, sildenafil citrate is used at an effective dose of 10 mg, which is made with sugar-coat in tablets by conventional process, and is used to achieve antineoplastic effects.

### IMPLEMENTATION EXAMPLE 2

As for the application of sildenafil of a low dose as an antineoplastic agent, the chemical name of mentioned sildenafil is 1-[[3-(6,7-Dihydro-1-methyl-7-oxo-3-propyl-1H-pyrazolo[4,3-d]pyrimidin-5-yl)-4-ethoxyphenyl]sulfonyl]-4-methylpiperazine citrate. The chemical structure is shown in FIG. 1 and the oral dosage of sildenafil is 0.04˜0.5 mg per 1 kg of body weight, or 2˜25 mg for an adult with body weight of 50 kg. FIG. 2 is a schematic diagram of the antineoplastic dose-window of sildenafil by mg.

For oral administration, sildenafil citrate is used at an effective dose of 20 mg, which is made with sugar-coat in tablets by conventional process, and is used to achieve antineoplastic effects.

### IMPLEMENTATION EXAMPLE 3

As for the application of sildenafil of a low dose as an antineoplastic agent, the chemical name of mentioned sildenafil is 1-[[3-(6,7-Dihydro-1-methyl-7-oxo-3-propyl-1H-pyrazolo[4,3-d]pyrimidin-5-yl)-4-ethoxyphenyl]sulfonyl]-4-methylpiperazine citrate. The chemical structure is shown in FIG. 1 and the oral dosage of sildenafil is 0.04˜0.5 mg per 1 kg of body weight, or 2˜25 mg for an adult with body weight of 50 kg. FIG. 2 is a schematic diagram of the antineoplastic dose-window of sildenafil by mg.

For oral administration, sildenafil citrate is used at an effective dose of 5 mg, which is made with sugar-coat in tablets by conventional process, and is used to achieve antineoplastic effects.

### IMPLEMENTATION EXAMPLE 4

As for the application of sildenafil of a low dose as an antineoplastic agent, the chemical name of mentioned sildenafil is 1-[[3-(6,7-Dihydro-1-methyl-7-oxo-3-propyl-1H-pyrazolo[4,3-d]pyrimidin-5-yl)-4-ethoxyphenyl]sulfonyl]-4-methylpiperazine citrate. The chemical structure is shown in FIG. 1 and the oral dosage of sildenafil is 0.04˜0.5 mg per 1 kg of body weight, or 2˜25 mg for an adult with body weight of 50 kg. FIG. 2 is a schematic diagram of the antineoplastic dose-window of sildenafil by mg.

For oral administration, sildenafil citrate is used at an effective dose of 10 mg, which is made in powder by conventional process, and is used to achieve antineoplastic effects.

### IMPLEMENTATION EXAMPLE 5

As for the application of sildenafil of a low dose as an antineoplastic agent, the chemical name of mentioned sildenafil is 1-[[3-(6,7-Dihydro-1-methyl-7-oxo-3-propyl-1H-pyrazolo[4,3-d]pyrimidin-5-yl)-4-ethoxyphenyl]sulfonyl]-4-methylpiperazine citrate. The chemical structure is shown in FIG. 1 and the oral dosage of sildenafil is 0.04˜0.5 mg per 1 kg of body weight, or 2˜25 mg for an adult with body weight of 50 kg. FIG. 2 is a schematic diagram of the antineoplastic dose-window of sildenafil by mg.

For oral administration, sildenafil citrate is used at an effective dose of 5 mg, which is made in powder by conventional process, and is used to achieve antineoplastic effects.

### IMPLEMENTATION EXAMPLE 6

As for the application of sildenafil of a low dose as an antineoplastic agent, the chemical name of mentioned sildenafil is 1-[4-ethyl-3-[5-(6,7-Two-methyl-7-oxygen generation-propyl H pyrazole and [4,3d] pyrimidine)] Benzene sulfonyl-4-biphenyl methyl piperazine citrate. The chemical structure is shown in FIG. 1 and the oral dosage of sildenafil is 0.04˜0.5 mg per 1 kg of body weight, or 2˜25 mg for an adult with body weight of 50 kg. FIG. 2 is a schematic diagram of the antineoplastic dose-window of sildenafil by mg.

For oral administration, sildenafil citrate is used at an effective dose of 2 mg, which is made in powder by conventional process, and is used to achieve antineoplastic effects.

### IMPLEMENTATION EXAMPLE 7

As for the application of sildenafil of a low dose as an antineoplastic agent, the chemical name of mentioned sildenafil is 1-[[3-(6,7-Dihydro-1-methyl-7-oxo-3-propyl-1H-pyrazolo[4,3-d]pyrimidin-5-yl)-4-ethoxyphenyl]sulfonyl]-4-methylpiperazine citrate. The chemical structure is shown in FIG. 1 and the oral dosage of sildenafil is 0.04˜0.5 mg per 1 kg of body weight, or 2˜25 mg for an adult with body weight of 50 kg. FIG. 2 is a schematic diagram of the antineoplastic dose-window of sildenafil by mg.

For oral administration, sildenafil citrate is used at an effective dose of 10 mg, which is made in capsules by conventional process, and is used to achieve antineoplastic effects.

### IMPLEMENTATION EXAMPLE 8

As for the application of sildenafil of a low dose as an antineoplastic agent, the chemical name of mentioned sildenafil is 1-[[3-(6,7-Dihydro-1-methyl-7-oxo-3-propyl-1H-pyrazolo[4,3-d]pyrimidin-5-yl)-4-ethoxyphenyl]sulfonyl]-4-methylpiperazine citrate. The chemical structure is shown in FIG. 1 and the oral dosage of sildenafil is 0.04˜0.5 mg per 1 kg of body weight, or 2˜25 mg for an adult with body weight of 50 kg. FIG. 2 is a schematic diagram of the antineoplastic dose-window of sildenafil by mg.

For oral administration, sildenafil citrate is used at an effective dose of 20 mg, which is made in capsules by conventional process, and is used to achieve antineoplastic effects.

### IMPLEMENTATION EXAMPLE 9

As for the application of sildenafil of a low dose as an antineoplastic agent, the chemical name of mentioned sildenafil is 1-[[3-(6,7-Dihydro-1-methyl-7-oxo-3-propyl-1H-pyrazolo[4,3-d]pyrimidin-5-yl)-4-ethoxyphenyl]sulfonyl]-4-methylpiperazine citrate. The chemical structure is shown in FIG. 1 and the oral dosage of sildenafil is 0.04˜0.5 mg per 1 kg of body weight, or 2˜25 mg for an adult with body weight of 50 kg. FIG. 2 is a schematic diagram of the antineoplastic dose-window of sildenafil by mg.

For oral administration, sildenafil citrate is used at an effective dose of 5 mg, which is made in capsules by conventional process, and is used to achieve antineoplastic effects.

### IMPLEMENTATION EXAMPLE 10

As for the application of sildenafil of a low dose as an antineoplastic agent, the chemical name of mentioned sildenafil is 1-[[3-(6,7-Dihydro-1-methyl-7-oxo-3-propyl-1H-pyrazolo[4,3-d]pyrimidin-5-yl)-4-ethoxyphenyl]sulfonyl]-4-methylpiperazine citrate. The chemical structure is shown in FIG. 1 and the oral dosage of sildenafil is 0.04˜0.5 mg per 1 kg of body weight, or 2˜25 mg for an adult with body weight of 50 kg. FIG. 2 is a schematic diagram of the antineoplastic dose-window of sildenafil by mg.

For oral administration, sildenafil citrate is used at an effective dose of 10 mg, which is made in oral liquid by conventional process, and is used to achieve antineoplastic effects.

### IMPLEMENTATION EXAMPLE 11

As for the application of sildenafil of a low dose as an antineoplastic agent, the chemical name of mentioned sildenafil is 1-[[3-(6,7-Dihydro-1-methyl-7-oxo-3-propyl-1H-pyrazolo[4,3-d]pyrimidin-5-yl)-4-ethoxyphenyl]sulfonyl]-4-methylpiperazine citrate. The chemical structure is shown in FIG. 1 and the oral dosage of sildenafil is 0.04˜0.5 mg per 1 kg of body weight, or 2˜25 mg for an adult with body weight of 50 kg. FIG. 2 is a schematic diagram of the antineoplastic dose-window of sildenafil by mg.

For oral administration, sildenafil citrate is used at an effective dose of 20 mg, which is made in oral liquid by conventional process, and is used to achieve antineoplastic effects.

### IMPLEMENTATION EXAMPLE 12

As for the application of sildenafil of a low dose as an antineoplastic agent, the chemical name of mentioned sildenafil is 1-[[3-(6,7-Dihydro-1-methyl-7-oxo-3-propyl-1H-pyrazolo[4,3-d]pyrimidin-5-yl)-4-ethoxyphenyl]sulfonyl]-4-methylpiperazine citrate. The chemical structure is shown in FIG. 1 and the oral dosage of sildenafil is 0.04˜0.5 mg per 1 kg of body weight, or 2˜25 mg for an adult with body weight of 50 kg. FIG. 2 is a schematic diagram of the antineoplastic dose-window of sildenafil by mg.

For oral administration, sildenafil citrate is used at an effective dose of 5 mg, which is made in oral liquid by conventional process, and is used to achieve antineoplastic effects.

### IMPLEMENTATION EXAMPLE 13

As for the application of sildenafil of a low dose as an antineoplastic agent, the chemical name of mentioned sildenafil is 1-[[3-(6,7-Dihydro-1-methyl-7-oxo-3-propyl-1H-pyrazolo[4,3-d]pyrimidin-5-yl)-4-ethoxyphenyl]sulfonyl]-4-methylpiperazine citrate. The chemical structure is shown in FIG. 1 and the oral dosage of sildenafil is 0.04˜0.5 mg per 1 kg of body weight, or 2˜25 mg for an adult with body weight of 50 kg. FIG. 2 is a schematic diagram of the antineoplastic dose-window of sildenafil by mg.

For oral administration, sildenafil citrate is used at an effective dose of 2 mg, which is made in oral liquid by conventional process, and is used to achieve antineoplastic effects.

### IMPLEMENTATION EXAMPLE 14

As for the application of sildenafil of a low dose as an antineoplastic agent, the chemical name of mentioned sildenafil is 1-[[3-(6,7-Dihydro-1-methyl-7-oxo-3-propyl-1H-pyrazolo[4,3-d]pyrimidin-5-yl)-4-ethoxyphenyl]sulfonyl]-4-methylpiperazine citrate. The chemical structure is shown in FIG. 1 and the injection dosage of sildenafil is 0.04˜0.2 mg per 1 kg of body weight, or 2˜10 mg for an adult with body weight of 50 kg. FIG. 2 is a schematic diagram of the antineoplastic dose-window of sildenafil by mg.

For subcutaneous administration, sildenafil citrate is used at an effective dose of 10 mg, which is made in injection agents by conventional process, and is subcutaneously injected to achieve antineoplastic effects.

### IMPLEMENTATION EXAMPLE 15

As for the application of sildenafil of a low dose as an antineoplastic agent, the chemical name of mentioned sildenafil is 1-[[3-(6,7-Dihydro-1-methyl-7-oxo-3-propyl-1H-pyrazolo[4,3-d]pyrimidin-5-yl)-4-ethoxyphenyl]sulfonyl]-4-methylpiperazine citrate. The chemical structure is shown in FIG. 1 and the injection dosage of sildenafil is 0.04˜0.2 mg per 1 kg of body weight, or 2˜10 mg for an adult with body weight of 50 kg. FIG. 2 is a schematic diagram of the antineoplastic dose-window of sildenafil by mg.

For subcutaneous administration, sildenafil citrate is used at an effective dose of 5 mg, which is made in injection agents by conventional process, and is subcutaneously injected to achieve antineoplastic effects.

### IMPLEMENTATION EXAMPLE 16

As for the application of sildenafil of a low dose as an antineoplastic agent, the chemical name of mentioned sildenafil is 1-[[3-(6,7-Dihydro-1-methyl-7-oxo-3-propyl-1H-pyrazolo[4,3-d]pyrimidin-5-yl)-4-ethoxyphenyl]sulfonyl]-4-methylpiperazine citrate. The chemical structure is shown in FIG. 1 and the injection dosage of sildenafil is 0.04˜0.2 mg per 1 kg of body weight, or 2˜10 mg for an adult with body weight of 50 kg. FIG. 2 is a schematic diagram of the antineoplastic dose-window of sildenafil by mg.

For subcutaneous administration, sildenafil citrate is used at an effective dose of 2 mg, which is made in injection agents by conventional process, and is subcutaneously injected to achieve antineoplastic effects.

### IMPLEMENTATION EXAMPLE 17

As for the application of sildenafil of a low dose as an antineoplastic agent, the chemical name of mentioned sildenafil is 1-[[3-(6,7-Dihydro-1-methyl-7-oxo-3-propyl-1H-pyrazolo[4,3-d]pyrimidin-5-yl)-4-ethoxyphenyl]sulfonyl]-4-methylpiperazine citrate. The chemical structure is shown in FIG. 1 and the injection dosage of sildenafil is 0.04˜0.2 mg per 1 kg of body weight, or 2˜10 mg for an adult with body weight of 50 kg. FIG. 2 is a schematic diagram of the antineoplastic dose-window of sildenafil by mg.

For intravenous administration, sildenafil citrate is used at an effective dose of 5 mg, which is made in injection agents by conventional process, and is intravenously injected to achieve antineoplastic effects.

### IMPLEMENTATION EXAMPLE 18

As for the application of sildenafil of a low dose as an antineoplastic agent, the chemical name of mentioned sildenafil is 1-[[3-(6,7-Dihydro-1-methyl-7-oxo-3-propyl-1H-pyrazolo[4,3-d]pyrimidin-5-yl)-4-ethoxyphenyl]sulfonyl]-4-methylpiperazine citrate. The chemical structure is shown in FIG. 1 and the injection dosage of sildenafil is 0.04˜0.2 mg per 1 kg of body weight, or 2˜10 mg for an adult with body weight of 50 kg. FIG. 2 is a schematic diagram of the antineoplastic dose-window of sildenafil by mg.

For intravenous administration, sildenafil citrate is used at an effective dose of 2 mg, which is made in injection agents by conventional process, and is intravenously injected to achieve antineoplastic effects.

### IMPLEMENTATION EXAMPLE 19

As for externally applied agents, sildenafil citrate is used at an effective dose of 1% by mass weight with lipid-soluble medium of 99%, which is made in ointment by conventional process, and is externally used to achieve antineoplastic effects.

### IMPLEMENTATION EXAMPLE 20

As for externally applied agents, sildenafil citrate is used at an effective dose of 0.1% by mass weight with lipid-soluble medium of 99.9%, which is made in ointment by conventional process, and is externally used to achieve antineoplastic effects.

### IMPLEMENTATION EXAMPLE 21

As for externally applied agents, sildenafil citrate is used at an effective dose of 0.01% by mass weight with lipid-soluble medium 99.99%, which is made in ointment by conventional process, and is externally used to achieve antineoplastic effects.

The implementation examples 1-21 of the invention, in which the daily oral dosage of 0.04˜0.5 mg per 1 kg of body weight, or 2˜25 mg for an adult with body weight of 50 kg, or injection dosage of 0.04˜0.2 mg per 1 kg of body weight or 2˜10 mg for an adult with body weight of 50 kg, or externally used dosage of less than 1% by mass weight of sildenafil citrate, are taken to achieve antineoplastic effects. It can inhibit tumor growth with inhibition rates of more than 50% without significant toxic side effects, and thereby effectively control tumor growth and improve the treatment of tumors.

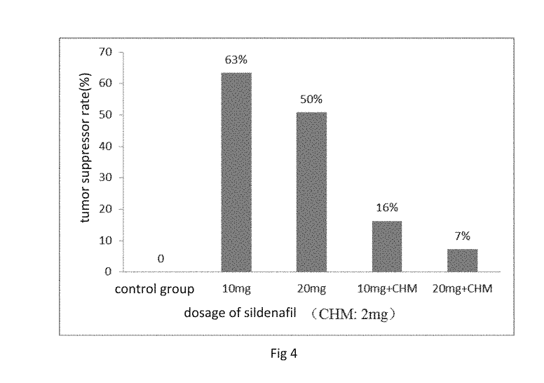

It should be noticed that the above external dosage is based on the total amount of sildenafil within the dose-window of 2˜25 mg. In the mouse transplanted tumor experiments, sildenafil citrate solution (dissolved in 25% ethanol) gavaged at 10 mg/kg/days for 36 days significantly inhibited tumor growth, with an inhibition rate of 68%, and the mice during the experimental period showed no significant or observable adverse reactions. However, when the dose was 50 mg/kg/day, sildenafil citrate had no obvious inhibitory effects on the growth of transplanted tumor in mice, as shown in FIG. 3.

The anti-tumor effect of low-dose sildenafil has an optimal dose-window, located between 0.04˜0.5 mg per 1 kg of body weight in humans, or 2˜25 mg for an adult with body weight of 50 kg, which is a low dose range of the drug with best anti-tumor efficacy and safety: the least adverse effects on cancer patients, but the best anti-tumor effects. Below or above this dose range (0.04˜0.5 mg per 1 kg of body weight, or 2-25 mg for an adult with body weight of 50 kg) will significantly weaken its anti-tumor efficacy. Since this dose range is significantly less than the normal dosage used for male erectile dysfunction, it is known as “low dose” sildenafil. In comparison with the normal dosage (25-100 mg for an adult), low dose sidenafil is not only able to significantly improve the anti-tumor efficacy of sildenafil, but also to avoid the side effects in the cancer patients caused by sildenafil.

Depending on the age, body weight and physical conditions of tumor patients, the optimal dosage of sildenafil as an antineoplastic agent will change in this dose range. For example, for the average adult patients (weight around 60 kg), the optimal oral dose of sildenafil is 10 mg.

The antitneoplastic mechanism of low dose sildenafil is not yet clear. Cell experiment results showed that sildenafil of high concentration (200 μM) directly inhibited the growth of cultured tumor cells, and thus the mechanism may be the stop growth of tumor cells in the G1 period resulted from inhibition of the cycle protein (cyclin) and cell cycle-dependent protein kinase (CDK) by the drug. In addition, sildenafil of higher concentration (300 μM) can also induce the production of reactive oxygen species (ROS) in cultured tumor cells, resulting in tumor cell apoptosis. However, after an adult patient taking sildenafil citrate tablet of 100 mg, the highest concentration of sildenafil in the blood is 440 ng/ml, or 0.66 μM, indicating that sildenafil at this dose is far lower in the tissue than the above concentrations required for the antineoplastic mechanism. Therefore, the antineoplastic efficacy of low dose sildenafil in vivo does not belong to the above antitumor mechanism.

Other studies have also shown that lower concentration (2 μM) of sildenafil can stimulate the expression of nuclear factor inhibitory protein IkB nitro-tyrosine and apoptotic gene ligand Fas-L in human hepatocellular carcinoma cells (HEPG2), and thus promotes tumor cell apoptosis. If the concentration of sildenafil in the blood after an adult patient orally takes low dose (2-10 mg) of the drug is considered, which should be lower than 0.1 μM in tissues, this low dose sildenafil may not directly inhibit the growth of tumor cells in vivo as seen in tumor cell experiments, and the antineoplastic mechanism for low dose oral sildenafil is not the same as that for tumor cell inhibition in vitro. Therefore, the antineoplastic activity of low dose sildenafil in vivomay not be a direct effect of the drug on the tumor cells.

FIG. 4 is a schematic diagram of the antagonistic effect of chlorpheniramine malate on the antitumor activity of sildenafil. It refers in particular to the inhibitory effects of sildenafil alone ontransplanted tumor after two week's administration of the drug at dose of 2 mg/kg/day, and the antagonistic effect when sildenafil was combined with chlorpheniramine malatein mice. In the figure, CHM refers to chlorpheniramine malate.

In the mouse transplanted tumor tests, it was demonstrated that the antineoplastic efficacy of low dose sildenafil was offset by the anti-allergic drug chlorphenamine malate. When the tumor-bearing mice were gavaged with 10 mg/kg/day or 20 mg/kg/day of sildenafil citrate for 2 weeks, the tumor inhibition rates were 63% and 50 % respectively, but if the drug was combined with 2 mg/kg/day of chlorphenamine malate, the tumor inhibition rates were reduced to 16% and 7%, respectively. As shown in FIG. 4, it is indicated that chlorphenamine malate significantly counteracted the antineoplastic activity of low dose sildenafil. Therefore, the antineoplastic activity of low dose sildenafil in vivo does not directly affect the tumor cells, but involves regulation of the immune system in the body, by increasing the functions of immune cells to achieve the antineoplastic effects. Because the immune cells such as lymphocytes, granulocytes and mononuclear cells have H1 receptors, and the H1 receptor antagonist chlorphenamine malate inhibits the immune response, this may explain why chlorphenamine malate counteracts the antineoplastic efficacy of low dose sildenafil in vivo.

The technical proposal provided by the implementation examples of the invention has been described in details. This paper expounds the theory for the implementation examples and the implementation methods with specific cases. The explanation of above examples is also applicable to the understanding of the theory of the invention; at the same time, for the general technical personnel in this field, according to the embodiment of the invention, there will be changes in the specific way of implementation and scope of application. In summary, the contents of this specification should not be construed as limitations of the invention.

1. An application of sildenafil at low dosage as an anticancer agent, with its characteristics that the chemical name is 1-[[3-(6,7-Dihydro-1-methyl-7-oxo-3-propyl-1H-pyrazolo[4,3-d]pyrimidin-5-yl)-4-ethoxyphenyl]sulfonyl]-4-methylpiperazine citrate, the formula is C₂₂H₃₀N₆O₄S.

2. According to the application of sildenafil at low dosage as an anticancer agent mentioned in claim 1, with its characteristic that the application methods of sildenafil include oral administration, injection and external use.

3. According to the application of sildenafil at low dosage as an anticancer agent mentioned in claim 1, with its characteristic that sildenafil can be prepared as oral liquid, powder, tablet, capsule, injection or ointment.

4. According to the application of sildenafil at low dosage as an anticancer agent mentioned in claim 2, with its characteristic that the oral dosage of sildenafil is 0.04˜0.5 mg per 1 kg of body weight, or 2˜25 mg for an adult with body weight of 50 kg.

5. According to the application of sildenafil at low dosage as an anticancer agent mentioned in claim 2, with its characteristic that the injection dosage of sildenafil is 0.04˜0.2 mg per 1 kg of body weight, or 2˜10 mg for an adult with body weight of 50 kg.

6. According to the application of sildenafil at low dosage as an anticancer agent mentioned in claim 2, with its characteristic that for external use, the mass percentage of sildenafil is lower than 1%.

7. According to the application of sildenafil at low dosage as an anticancer agent mentioned in claim 6, with its characteristic that the formula of externally used ointment of sildenafil is as follows: sildenafil of a mass percentage of 0.01˜1% and the oil soluble medium of a mass percentage of 99˜99.99%.
